(19) United States
(12) Patent Application Publication
(10) Pub. No.: US 2006/0167056 A1
(43) Pub. Date: Jul. 27, 2006

(54) Polymorphs of {5-[3-(4,6-Difluoro-1H-benzoimidazol-2-yl)-1H-indazol-5-yl)-4-methyl-pyridin-3-ylmethyl}-ethyl-amine

(75) Inventors: Raymond Ronald Rynberg, Spring Valley, CA (US); Rongliang Chen, San Diego, CA (US)

Correspondence Address: AGOURON PHARMACEUTICALS, INC., 10777 SCIENCE CENTER DRIVE, SAN DIEGO, CA, 92121, US

(73) Assignee: AGOURON PHARMACEUTICALS, INC.

(21) Appl. No.: 11/281,295

(22) Filed: Nov. 16, 2005

Related U.S. Application Data
(60) Provisional application No. 60/629,010, filed on Nov. 17, 2004.
(60) Provisional application No. 60/672,623, filed on Apr. 18, 2005.

(51) Int. Cl. A61K 31/4439; C07D 403/14

(52) U.S. Cl. 514/338; 546/273.4

(57) ABSTRACT

The present invention relates to novel polymorphic forms of of 3{5-[3-(4,6-Difluoro-1H-benzoimidazol-2-yl)-1H-indazol-5-yl]-4-methyl-pyridin-3-ylmethyl}-ethyl-amine, and to processes for their preparation. Such polymorphic forms may be a component of a pharmaceutical composition and may be used to treat a hyperproliferative disorder or a mammalian disease condition mediated by protein kinase activity.

This application claims the benefit of U.S. Provisional Application No. 60/629,010 filed on Nov. 17, 2004, the contents of which are hereby incorporated by reference in their entirety.

### FIELD OF THE INVENTION

This invention relates to novel polymorphic forms of {5-[3-(4,6-Difluoro-1H-benzoimidazol-2-yl)-1H-indazol-5-yl]-4-methyl-pyridin-3-ylmethyl}-ethyl-amine, and methods for their preparation. The invention is also directed to pharmaceutical compositions containing at least one polymorphic form and to the therapeutic or prophylactic use of such compositions.

### BACKGROUND OF THE INVENTION

The compound {5-[3-(4,6-Difluoro-1H-benzoimidazol-2-yl)-1H-indazol-5-yl]-4-methyl-pyridin-3-ylmethyl}-ethyl-amine (also referred to as “Compound 1”), 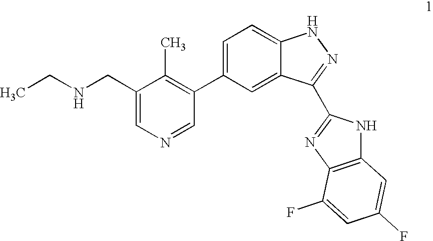
as well as pharmaceutically acceptable salts and solvates thereof, is described in U.S. patent application Ser. No. 10/866,069, filed 10 Jun. 2004, the disclosure of which is hereby incorporated in its entirety. This compound is a protein kinase inhibitor and represents a synthetic, small molecule inhibitor capable of modulating cell cycle control.

Cell proliferation occurs in response to various stimuli and may stem from de-regulation of the cell division cycle (or cell cycle), the process by which cells multiply and divide. Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and thus hyperproliferation, could be used to treat various disease states associated with uncontrolled or unwanted cell proliferation.

At the cellular level, de-regulation of signaling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins. Among the candidate regulatory proteins, protein kinases are a family of enzymes that catalyze phosphorylation of the hydroxyl group of specific tyrosine, serine or threonine residues in proteins. In particular, cyclin-dependent kinases (“CDKs”) are serine-threonine protein kinases that play critical roles in regulating the transitions between different phases of the cell-cycle, such as the progression from a quiescent stage in G₁ (the gap between mitosis and the onset of DNA replication for a new round of cell division) to S (the period of active DNA synthesis), or the progression from G₂ to M phase, in which active mitosis and cell-division occurs. CDK complexes are formed through association of a regulatory cyclin subunit (e.g., cyclin A, B1, B2, D1, D2, D3, and E) and a catalytic kinase subunit (e.g., CDK1, CDK2, CDK4, CDK5, and CDK6). As the name implies, CDKs display an absolute dependence on the cyclin subunit in order to phosphorylate their target substrates, and different kinase/cyclin pairs function to regulate progression through specific phases of the cell-cycle.

There is thus a need for effective inhibitors of protein kinases. Moreover, as is understood by those skilled in the art, it is desirable for kinase inhibitors to possess physical properties amenable to reliable formulation. These properties include stability to heat, moisture, and light.

Crystalline polymorphs are different crystalline forms of the same compound. The term polymorph may or may not include other solid state molecular forms including hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such the X-ray diffraction characteristics of crystals or powders. A different polymorph, for example, will in general diffract at a different set of angles and will give different values for the intensities. Therefore X-ray powder diffraction can be used to identify different polymorphs, or a solid form that comprises more than one polymorph, in a reproducible and reliable way.

Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

### SUMMARY OF THE INVENTION

In one aspect, the present invention provides at least two polymorphic forms and an amorphous form of Compound 1.

In one embodiment, the invention provides a substantially pure polymorph of 3{5-[3-(4,6-Difluoro-1H-benzoimidazol-2-yl)-1H-indazol-5-yl]-4-methyl-pyridin-3-ylmethyl}-ethyl-amine (Compound 1), represented by Formula 1 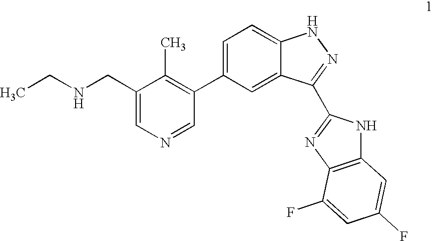
wherein the crystalline form is a substantially pure polymorph of Form B. More particularly, polymorph form B has a powder X-ray diffraction pattern comprising peaks at diffraction angles (2θ) of 10.5±0.1, 12.0±0.1 and 22.3±0.1. Even more particularly, polymorph Form B has a powder X-ray diffraction pattern comprising peaks at diffraction angles (2θ) of 10.5±0.1, 12.0±0.1, 12.5±0.1, 13.6±0.1 and 22.3±0.1. Even more particularly, polymorph Form B has a powder X-ray diffraction pattern comprising peaks at diffraction angles (2θ) of 10.5±0.1, 12.0±0.1, 12.5±0.1, 13.6±0.1, 15.4±0.1 and 22.3±0.1.

In another aspect, polymorph Form B is characterized by Raman shifts (cm⁻¹) at 717±1, 1142±1 and 1514±1. More particularly, polymorph Form B is further characterized by Raman shifts (cm⁻¹) at 717±1, 1142±1, 1311±1, 1333±1 and 1514±1.

In another aspect, polymorph Form B is characterized by ¹³C solid state NMR shifts (ppm) at 118.8±1, 124.6±1, and 129.6±1. More particularly, polymorph Form B is further characterized by ¹³C solid state NMR shifts (ppm) at 118.8±1, 124.6±1, 129.6±1, 155.8±1 and 157.7±1. In still another aspect, polymorph Form B is characterized by ¹⁹F solid state NMR shifts (ppm) at −113.9±1, −118.6±1, −124.3±1 and −126.2±1.

In another embodiment of the invention, the invention provides a substantially pure form of Compound 1, wherein the crystalline form is a substantially pure polymorph of Form C. More particularly, polymorph Form C, has a powder X-ray diffraction pattern comprising peaks at diffraction angles (2θ) of 6.7±0.1 and 8.1±0.1. Still more particularly, polymorph Form C has a powder X-ray diffraction pattern comprising peaks at diffraction angles (2θ) of 6.7±0.1, 8.1±0.1 and 23.4±0.1. Even more particularly, polymorph Form C has a powder X-ray diffraction pattern comprising peaks at diffraction angles (2θ) of 6.7±0.1, 8.1±0.1, 14.7±0.1 and 23.4±0.1.

In another aspect of the invention, polymorph Form C is characterized by Raman shifts (cm⁻¹) of 1340±1, 1434±1 and 1510±1. More particularly, polymorph Form C is further characterized by Raman shifts (cm⁻¹) of 1252±1, 1304±1, 1340±1, 1434±1 and 1510±1.

In yet another aspect of the invention, polymorph Form C is characterized by ¹³C solid state NMR shifts at 122.0±1, 135.7±1 and 139.6±1. More particularly, polymorph Form C is further characterized by ¹³C solid state NMR shifts at 122.0±1, 131.6±1, 135.7±1, 139.6±1 and 148.2±1. In yet another aspect of the invention, polymorph Form C is characterized by ¹⁹F solid state NMR shifts (ppm) at −114.1±1, −125.7±1, and −128.2±1.

In yet another embodiment of the invention, the invention provides an amorphous form of Compound 1, wherein there is no crystalline form as depicted by an X-ray powder diffraction pattern. In one aspect of the invention, the amorphous form of Compound 1 is characterized by having a powder X-ray diffraction pattern essentially the same as shown in FIG. 4A. In another aspect of the invention, the amorphous form of Compound 1 is characterized by Raman shifts (cm⁻¹) of 233±1 and 1580±1. In a further aspect of this embodiment, the amorphous form of Compound 1 is characterized by Raman shifts (cm⁻¹) of 233±1, 1249±1, and 1580±1. In a still further aspect of this embodiment, the amorphous form of Compound 1 is characterized by Raman shifts (cm⁻¹) of 233±1, 707±1, 1249±1, and 1580±1.

In still another embodiment of the invention, the invention provides a mixture comprising at least one of the polymorphic forms B or C and an amorphous form Compound 1. In still a further embodiment of the invention, the invention provides a mixture of polymorphic forms of 3{5-[3-(4,6-Difluoro-1H-benzoimidazol-2-yl)-1H-indazol-5-yl]-4-methyl-pyridin-3-ylmethyl}-ethyl-amine, comprising at least two of the following polymorphic forms A, B, or C. In another aspect, the invention relates to pharmaceutical compositions, each comprising a crystalline form of Compound 1. The invention also relates to a pharmaceutical composition comprising a mixture of at least two of any of the polymorphic forms. In particular, the invention provides a pharmaceutical composition comprising the crystalline form of polymorph Form B. In an alternative embodiment, the invention provides a pharmaceutical composition comprising the crystalline form of polymorph Form C. In yet another alternative embodiment, the invention provides a pharmaceutical composition comprising the amorphous form of Compound 1. In yet a further embodiment, the invention provides a pharmaceutical composition comprising a mixture of polymorph Forms B, C or the amorphous form.

In another aspect, the invention provides methods of treating a mammalian disease condition mediated by protein kinase activity, comprising administering to a mammal in need thereof a therapeutically effective amount of the pharmaceutical composition comprising any one of polymorphs B, C or the amorphous form. In a particular aspect of this embodiment, the method comprises administering a therapeutically effective amount of polymorph Form B.

In a further aspect, the invention provides methods of selectively inhibiting CDK kinase activity by administering to a patient in need thereof a therapeutically effective amount of polymorph compound of the invention. In a particular aspect of this embodiment, the method comprises administering a therapeutically effective amount of Form B polymorph.

The compounds of the invention may be used advantageously in combination with other known therapeutic agents. For example the polymorphic forms of Compound 1 may be co-administered with or one more other anti-tumor agents, anti-angiogenesis agents, signal transduction inhibitors and antiproliferative agents, which amounts are together effective in treating cellular proliferation.

In another embodiment, the invention provides a mixture of polymorphs of Compound 1, where the mixture comprises at least 50% of Form B.

In another embodiment, the invention provides a mixture of polymorphs of Compound 1, where the mixture comprises at least 60% of Form B.

In another embodiment, the invention provides a mixture of polymorphs of Compound 1, where the mixture comprises at least 70% of Form B.

In another embodiment, the invention provides a mixture of polymorphs of Compound 1, where the mixture comprises at least 80% of Form B.

In another embodiment, the invention provides a mixture of polymorphs of Compound 1, where the mixture comprises at least 90% of Form B.

In a further aspect, the invention provides methods of treating mycotic infection, malignancies or cancer as well as other diseases associated with unwanted angiogenesis or cellular proliferation, comprising administering to a patient in need thereof a therapeutically effective amount of polymorph compound of the invention. In a particular aspect of this embodiment, the method comprises administering a therapeutically effective amount of Form B polymorph.

The present invention is also directed to combination therapeutic methods of treating a hyperproliferative disorder, or a disease condition mediated by CDK activity, which comprises administering to a mammal in need thereof a therapeutically effective amount of a pharmaceutical composition which comprises any of the polymorphic forms, or pharmaceutical compositions discussed above, in combination with a therapeutically effective amount of one or more substances selected from anti-tumor agents, anti-angiogenesis agents, signal transduction inhibitors, and antiproliferative agents.

The term “active agent” or “active ingredient” refers to a polymorphic form of Compound 1, or to a solid form that comprises two or more polymorphic forms of Compound 1.

The term “ambient temperature” refers to a temperature condition typically encountered in a laboratory setting. This includes the approximate temperature range of about 20 to about 30° C.

The term “amorphous” refers to a non-crystalline form of a compound.

The term “aqueous base” refers to any organic or inorganic base. Aqueous bases include, by way of example only, metal bicarbonates, such as sodium bicarbonate, potassium carbonate, cesium carbonate, and the like.

The term “aromatic solvent” refers to an organic solvent possessing an aromatic moiety, including by way of example only, benzene, toluene, xylene isomers or mixtures thereof, and the like.

The term “chemical stability” refers to a type of stability in which a particular compound maintains its chemical integrity, and includes, but is not limited to, thermal stability, light stability, and moisture stability.

The term “detectable amount” refers to an amount or amount per unit volume that can be detected using conventional techniques, such as X-ray powder diffraction, Differential Scanning Calorimetry, HPLC, FT-IR, Raman spectroscopy, and the like.

The term “exposing to humidity” refers to the process of exposing a substance to water vapor in a humidor, humidity chamber, or any apparatus capable of controlling relative humidity. The term may also describe the process of exposing a substance to ambient humidity as during storage.

The term “hyperproliferative disorder” refers to abnormal cell growth that is independent of normal regulatory mechanisms (e.g., loss of contact inhibition), including the abnormal growth of normal cells and the growth of abnormal cells. This includes, but is not limited to, the abnormal growth of tumor cells (tumors), both benign and malignant. Examples of such benign proliferative diseases are psoriasis, benign prostatic hypertrophy, human papilloma virus (HPV), and restinosis. The term “hyperproliferative disorder” also refers to cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

The term “inert solvent” refers to any solvent or liquid component of a slurry that does not chemically react with other components in a solution or slurry. Inert solvents include, by way of example only, aprotic solvents such as aromatic solvents, ethyl acetate, acetone, methyl tert-butylether, dioxane, THF, and the like. Protic solvents include, by way of example only, methanol, ethanol, propanol isomers, butanol isomers and the like.

The term “mediated by CDK activity” refers to biological or molecular processes that are regulated, modulated, or inhibited by CDK protein kinase activity. For certain applications, inhibition of the protein kinase activity associated with CDK complexes, among others, and those which inhibit angiogenesis and/or inflammation are preferred. The present invention includes methods of modulating or inhibiting protein kinase activity, for example in mammalian tissue, by administering polymorphic forms of Compound 1. The activity of agents as anti-proliferatives is easily measured by known methods, for example by using whole cell cultures in an MTT assay. The activity of polymorphic forms of Compound 1 as mediators of protein kinase activity, such as the activity of kinases, may be measured by any of the methods available to those skilled in the art, including in vivo and/or in vitro assays.

The term “minimal amount” refers to the least amount of solvent required to completely dissolve a substance at a given temperature.

The term “pharmaceutically acceptable salt” refers to a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form pharmaceutically acceptable salts. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, para-toluene sulfonates (tosylates), formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The term “polymorph” refers to different crystalline forms of the same compound and includes, but is not limited to, other solid state molecular forms including hydrates (e.g., bound water present in the crystalline structure) and solvates (e.g., bound solvents other than water) of the same compound.

The term “peak intensities” refers to relative signal intensities within a given X-ray diffraction pattern. Factors which can affect the relative peak intensities are sample thickness and preferred orientation (i.e. the crystalline particles are not distributed randomly).

The term “peak positions” as used herein refers to X-ray reflection positions as measured and observed in X-ray powder diffraction experiments. Peak positions are directly related to the dimensions of the unit cell. The peaks, identified by their respective peak positions, have been extracted from the diffraction patterns for the various polymorphic Forms A, B, and C of Compound 1.

The term “PEG” refers to poly(ethylene glycol). PEG is commercially available having different ranges of polymer chain lengths and thus viscosities. PEG 400 is soluble in alcohols, acetone, benzene, chloroform, acetic acid, CCl₄, and water.

The term “pharmaceutically acceptable, carrier, diluent, or vehicle” refers to a material (or materials) that may be included with a particular pharmaceutical agent to form a pharmaceutical composition, and may be solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

The term “pharmaceutical composition” refers to a mixture of one or more of the compounds or polymorphs described herein, or physiologically/pharmaceutically acceptable salts or solvates thereof, with other chemical components, such as physiologically/pharmaceutically acceptable carriers and excipients. The purpose of a pharmaceutical composition is to facilitate administration of a compound to an organism.

The term “recrystallize” refers to the process of completely dissolving a solid in a first solvent with heating if necessary, and then inducing precipitation, usually by cooling the solution, or by adding a second solvent in which the solid is poorly soluble.

The term “relative intensity” refers to an intensity value derived from a sample X-ray diffraction pattern or a Raman shift pattern. The complete ordinate range scale for a diffraction pattern is assigned a value of 100. A peak having intensity falling between about 50% to about 100% on this scale intensity is termed very strong (vs); a peak having intensity falling between about 50% to about 25% is termed strong (s). Additional weaker peaks are present in typical diffraction patterns and are also characteristic of a given polymorph.

The term “slurry” refers to a solid substance suspended in a liquid medium, typically water or an organic solvent.

The term “separating from” refers to a step in a synthesis in which the desired agent is isolated from other non-desired agents, including, but not limited to any of the following steps: filtering, washing with extra solvent or water, drying with heat and or under vacuum.

The term “substantially pure” with reference to particular polymorphic forms of Compound 1 means the polymorphic form includes less than 10% by weight of impurities, including other polymorphic or amorphous forms of Compound 1. In one embodiment, the substantially pure polymorphic form includes less than 3% by weight of impurities, including other polymorphic or amorphous forms of Compound 1. In a further embodiment, substantially pure polymorphic form includes less than 1% by weight of impurities, including other polymorphic or amorphous forms of Compound 1. Such purity may be determined, for example, by X-ray powder diffraction.

An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

The term “therapeutically effective amount” refers to that amount of the compound or polymorph being administered which will relieve to some extent one or more of the symptoms of the disorder being treated. In reference to the treatment of cancer, a therapeutically effective amount refers to that amount which has at least one of the following effects:
(1) reducing the size of the tumor;
(2) inhibiting (that is, slowing to some extent, preferably stopping) tumor metastasis;
(3) inhibiting to some extent (that is, slowing to some extent, preferably stopping) tumor growth, and
(4) relieving to some extent (or, preferably, eliminating) one or more symptoms associated with the cancer.

The term “2 theta value” or “2θ” refers to the peak position based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2 theta (2θ).

The terms “treat”, “treating” and “treatment” refer to a method of alleviating or abrogating a hyperproliferative disorder and/or its attendant symptoms. With regard particularly to cancer, these terms simply mean that the life expectancy of an individual affected with a cancer will be increased or that one or more of the symptoms of the disease will be reduced.

The term “under vacuum” refers to typical pressures obtainable by a laboratory oil or oil-free diaphragm vacuum pump.

The term “X-ray powder diffraction pattern” refers to the experimentally observed diffractogram or parameters derived therefrom. X-Ray powder diffraction patterns are characterized by peak position (abscissa) and peak intensities (ordinate).

The term “xylenes” refers to any of the xylene isomers or a mixture thereof.

### BRIEF DESCRIPTION OF THE DRAWINGS

Having thus described the invention in general terms, reference will now be made to the accompanying drawings, wherein:

FIG. 1A is an X-ray powder diffraction diagram of polymorph Form A of the invention;

FIG. 1B is a Raman spectra of polymorph Form A of the invention;

FIG. 1C is a ¹³C solid state NMR spectra of polymorph Form A of the invention;

FIG. 1D is a ¹⁹F solid state NMR spectra of polymorph Form A of the invention;

FIG. 1E is a Differential Scanning Calorimetry (DSC) profile of polymorphic Form A of the invention (a typical profile displays endotherms with onset at 167-171° C. at a scan rate of 10° C./min);

FIG. 1F is a Thermal Gravimetric Analysis (TGA) profile of polymorph Form A;

FIG. 2A is an X-ray powder diffraction diagram of polymorph Form B of the invention;

FIG. 2B is a Raman spectra of polymorph Form B of the invention;

FIG. 2C is a ¹³C solid state NMR spectra of polymorph Form B of the invention;

FIG. 2D is a ¹⁹F solid state NMR spectra of polymorph Form B of the invention;

FIG. 2E is a DSC profile of polymorphic Form B of the invention (a typical profile displays endotherms with onset at 248-250° C. at a scan rate of 10° C./min);

FIG. 2F is a TGA profile of polymorph Form B;

FIG. 2G is a DSC profile indicating the conversion of polymorph Form A to polymorph Form B of the invention;

FIG. 3A is an X-ray powder diffraction diagram of polymorph Form C of the invention;

FIG. 3B is a Raman spectra of polymorph Form C of the invention;

FIG. 3C is a ¹³C solid state spectra of polymorph Form C of the invention;

FIG. 3D is a ¹⁹F solid state spectra of polymorph Form C of the invention;

FIG. 3E is a DSC profile of polymorph Form C of the invention;

FIG. 4A is an X-ray powder diffraction diagram of the amorphous form of the invention; and

FIG. 4B is a Raman spectrum of the amorphous form of the invention.

### DETAILED DESCRIPTION OF THE INVENTION

It has surprisingly been found that the substance Compound 1 can exist in more than one polymorphic crystalline form. These forms may be used in a formulated product for the treatment of hyperproliferative indications, including cancer. Each form may have advantage over the others in bioavailability, stability, or manufacturability. Crystalline polymorphic forms of Compound 1 have been discovered which are likely to be more suitable for bulk preparation and handling than other polymorphic forms. Processes for producing these polymorphic forms in high purity are described herein. Another object of the present invention is to provide a process for the preparation of each polymorphic form of Compound 1, substantially free from other polymorphic forms of Compound 1. Additionally it is an object of the present invention to provide pharmaceutical formulations comprising Compound 1 in different polymorphic forms as discussed above, and methods of treating hyperproliferative conditions by administering such pharmaceutical formulations.

I. Polymorphic Forms of Compound 1

The present invention provides several polymorphic crystalline forms of Compound 1. Each crystalline form of the compound can be characterized by one or more of the following: X-ray powder diffraction pattern (i.e., X-ray diffraction peaks at various diffraction angles (2θ)), melting point onset (and onset of dehydration for hydrated forms) as illustrated by endotherms of a differential scanning calorimetry (DSC) thermogram, Raman spectra, thermal gravimetric analysis (TGA), solid state ¹³C and ¹⁹F nuclear magnetic resonance (SSNMR) spectrum, aqueous solubility, light stability under International Conference on Harmonization (ICH) high intensity light conditions, and physical and chemical storage stability.

The X-ray powder diffraction pattern for each polymorph or amorphous form of Compound 1 was measured on a Bruker AXS D8-Discover diffractometer equipped with Cu K α radiation 1.54 Å X-ray radiation source operated at 40 kV and 40 mA. During analysis, the samples were analyzed from angles of 4 to 40 degrees (θ-2θ) using a general area detraction detector. The detector was set 30 cm from sample. The X-Ray diffraction peaks, characterized by peak positions and intensity assignments, have been extracted from the X-ray powder diffractogram of each of the polymorphic forms of Compound 1. One of skill in the art will appreciate that the peak positions (2θ) will show some inter-apparatus variability, typically as much as 0.1 degrees. Accordingly, where peak positions (2θ) are reported, one of skill in the art will recognize that such numbers are intended to encompass such inter-apparatus variability. Furthermore, where the crystalline forms of the present invention are described as having a powder X-ray diffraction pattern essentially the same as that shown in a given figure, the term “essentially the same” is also intended to encompass such inter-apparatus variability in diffraction peak positions. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only.

The solid state ¹³C and ¹⁹F nuclear magnetic resonance (SSNMR) spectrum for each polymorph form of the invention was acquired at 500 MHz using a CP-MAS solid-state NMR probe wherein chemical shift is expressed in parts per million (ppm). Due to different amounts of each material available, two probes with two spinner sizes were employed. If sufficient amount of material was available, approximately 80 mg of sample were tightly packed into a 4 mm ZrO spinner. Otherwise approximately 12 mg of sample were tightly packed into a 2.5 mm ZrO spinner. The spectra were collected at 295 K and ambient pressure using either the Bruker-Biospin 4 mm BL HFX or the Bruker-Biospin 2.5 mm BL CPMAS probe, positioned into a wide-bore Bruker-Biospin Avance DSX 500 MHz NMR spectrometer. The samples were positioned at the magic angle.

The ¹³C solid state spectra were collected using a proton decoupled cross-polarization magic angle spinning experiment (CPMAS). The samples were spun at 20 and 15 kHz using the 2.5 and 4 mm probes, respectively. The proton decoupling field of approximately 110 and 85 kHz was applied using the 2.5 and 4 mm probes, respectively. Approximately 8000 and 1800 scans were collected using the 2.5 and 4 mm probes, respectively. The recycle delay was adjusted to seven seconds. The spectra were referenced using an external standard of crystalline adamantane, setting its upfield resonance to 29.5 ppm.

The ¹⁹F solid state spectra were collected using a magic angle spinning (MAS) experiment. Spinning speed was adjusted to 35.0 kHz, corresponding to the maximum specified spinning speed for the 2.5 mm BL probe. Sixteen scans were collected on each sample. The spectra were acquired with recycle delay of 90 s. The spectra were referenced using an external sample of trifluoro-acetic acid (50% V/V in H₂O), setting its resonance to −76.54 ppm. A baseline correction was applied to correct for the ¹⁹F probe background signal.

Where sufficient material was available, ¹⁹F solid state spectra was collected using a spinning speed that was adjusted to 15.0 kHz, corresponding to the maximum specified spinning speed for the 4 mm BL HFX probe. Sixteen scans were collected on each sample. The spectra were acquired with a recycle delay of 90 s, set as five times the fluorine longitudinal relaxation time (¹⁹F T₁) measured independently by an inversion recovery experiment. Proton longitudinal relaxation times (¹H T₁) were calculated based on fluorine detected proton inversion recovery relaxation experiment. The spectra were referenced using an external sample of trifluoro-acetic acid (50% V/V in H₂O), setting its resonance to −76.54 ppm. The Raman spectrum for each polymorph form of the invention was expressed in terms of the Raman shift (cm⁻¹) and relative intensities as measured on a Kaiser Optical Systems Raman microprobe with a solid-state diode laser operating at 785 nm.

Different polymorphic forms of Compound 1 were also distinguished using differential scanning calorimetry (DSC). DSC measures the difference in heat energy between a solid sample and an appropriate reference with an increase in temperature. DSC thermograms are characterized by endotherms (indicating energy uptake) and also by exotherms (indicating energy release), typically as the sample is heated. The DSC thermographs were obtained using a TA Instrument DSC Q-1000 instrument at a scan rate of 10° C./min over a temperature range of 25-300° C. Samples were weighed into aluminum crucibles that were sealed and punctured with a single hole. The extrapolated onset of melting temperature and, where applicable, the onset of dehydration/desolvation temperature, were also calculated. Depending upon the rate of heating (i.e., the scan rate) at which the DSC analysis is conducted, the way the DSC on-set temperature is defined and determined, the calibration standard used, the instrument calibration, and the relative humidity and chemical purity of the sample, the endotherms exhibited by the compounds of the invention may vary (by about 0.01-5° C., for crystal polymorph melting and by about 0.01-20° C. for polymorph dehydration/desolvation) above or below the endotherms. For any given example, the observed endotherms may also differ from instrument to instrument; however, it will generally be within the ranges defined herein provided the instruments are calibrated similarly.

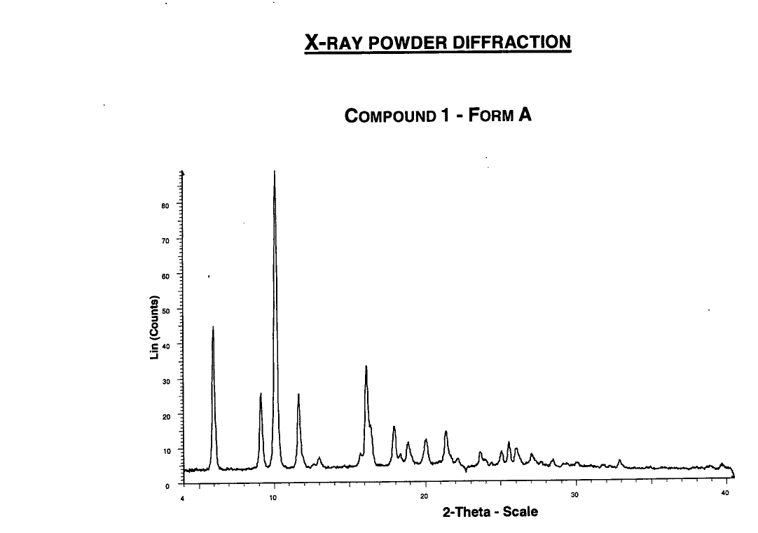

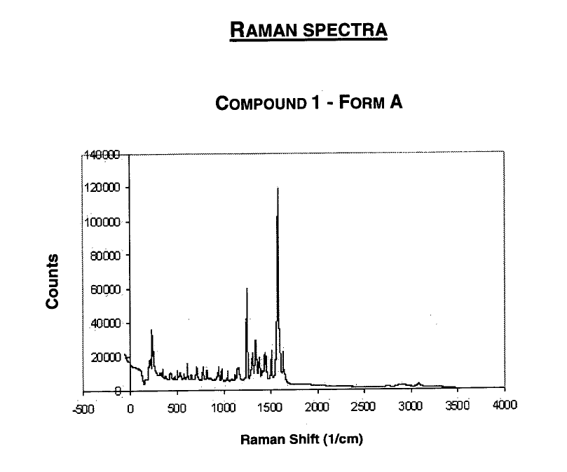

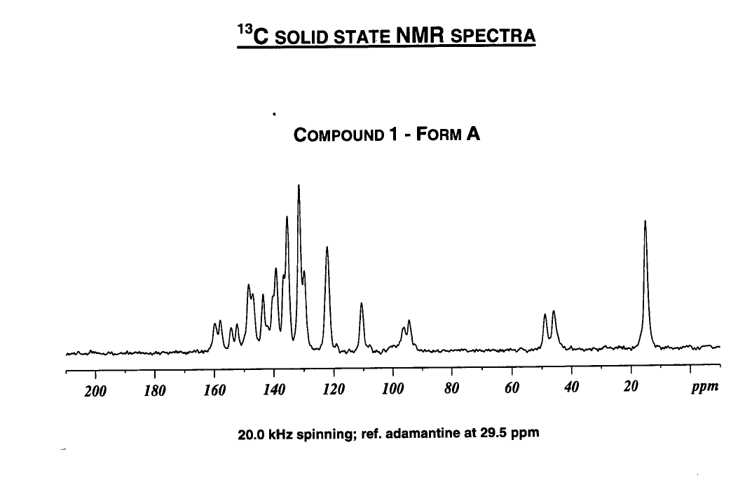

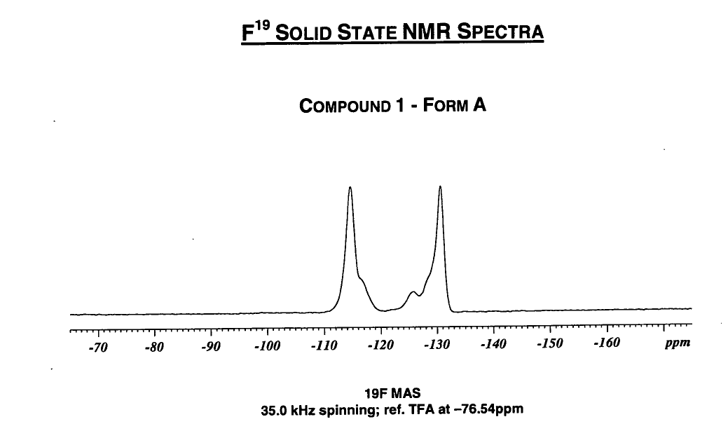

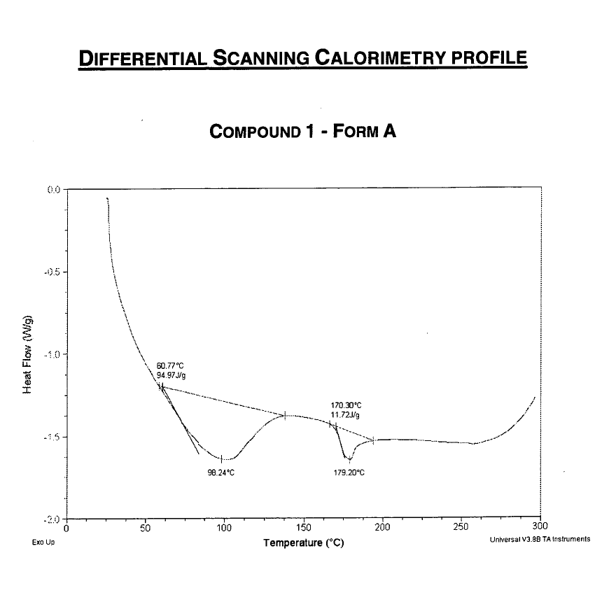

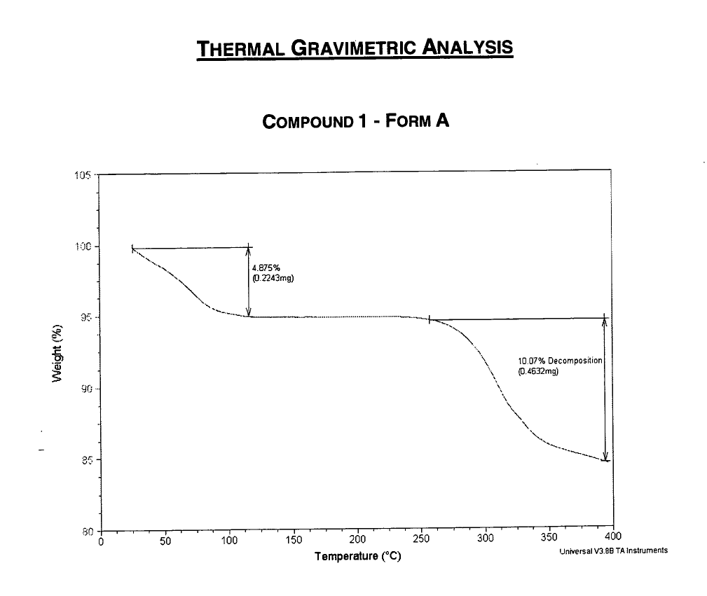

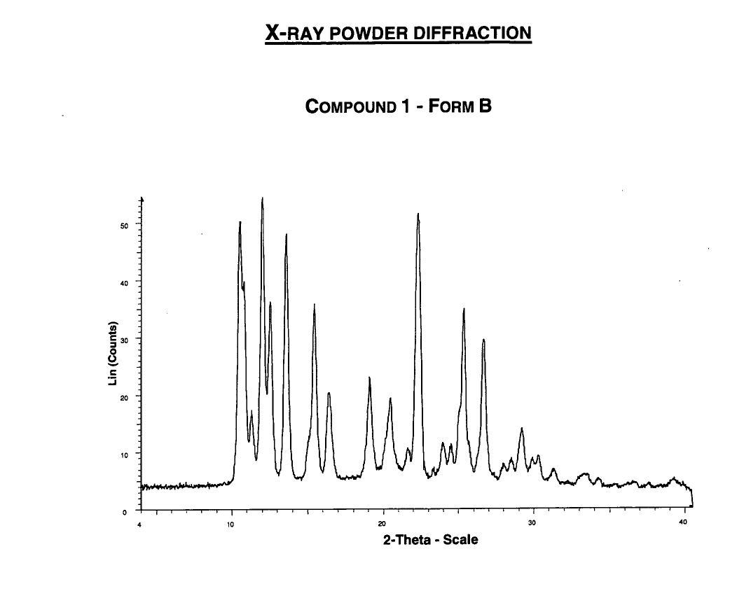

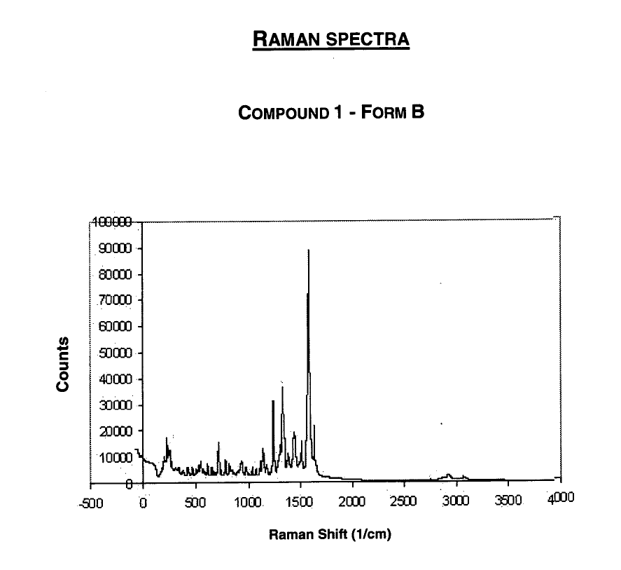

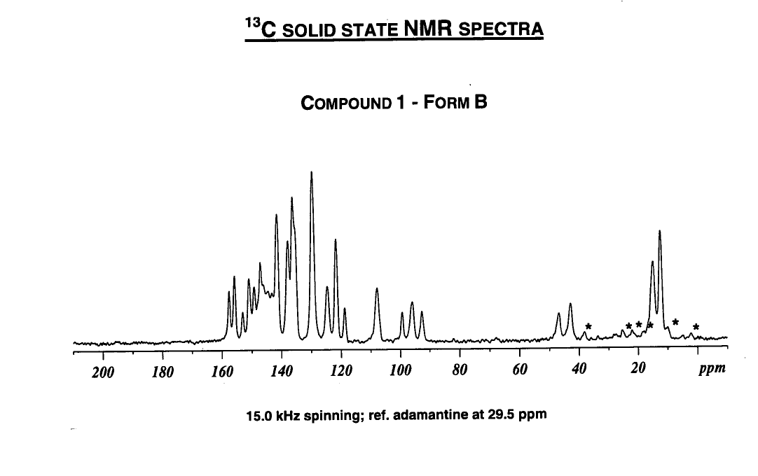

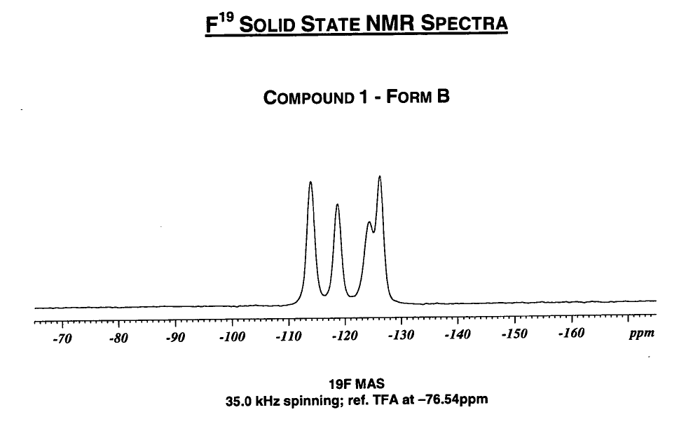

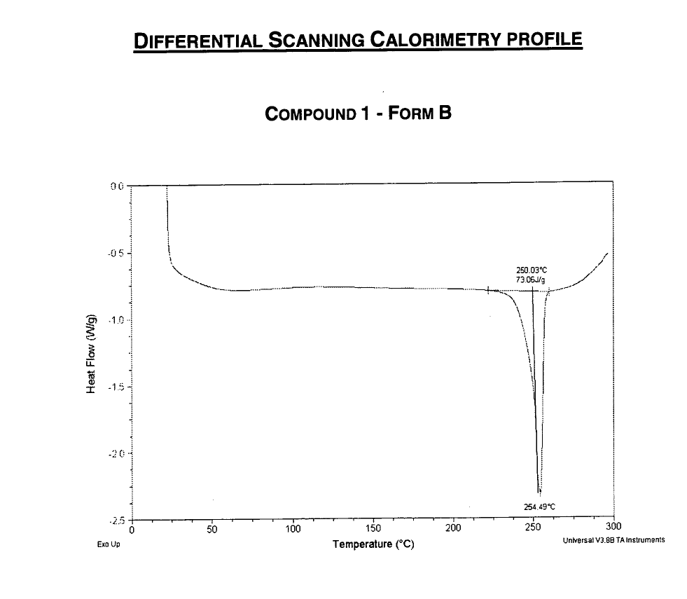

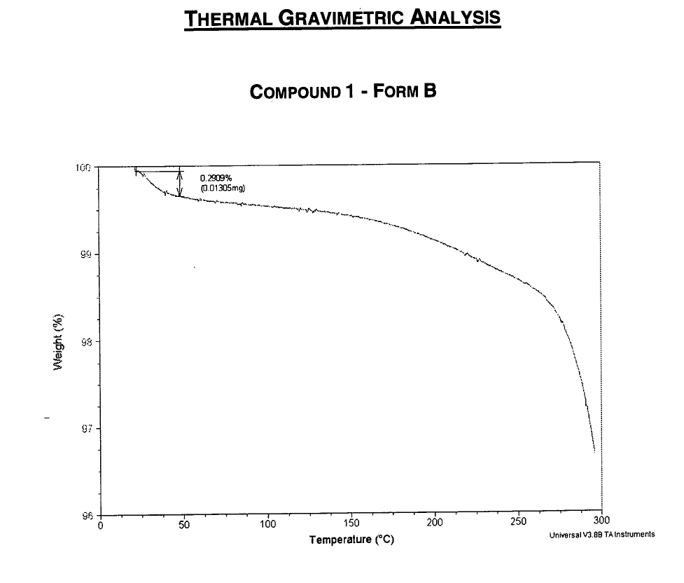

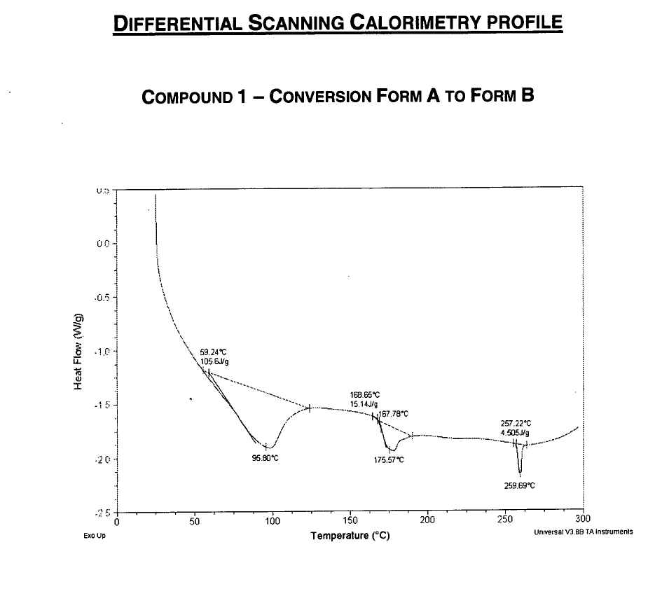

Different polymorphic forms of Compound 1 were also distinguished using thermal gravimetric analysis (TGA). TGAs were performed on a TA Instruments TGA Q 500 instrument. The temperature was ramped at 10° C./min to 400° C. for Form A (FIG. 1E) and 10° C./min to 300° C. for Form B (FIG. 2E). TGA is a testing procedure in which changes in weight of a specimen are recorded as the specimen is heated in air or in a controlled atmosphere such as nitrogen. Thermogravimetric curves (thermograms) provide information regarding solvent and water content and the thermal stability of materials.

Different polymorphic forms of Compound 1, may also be distinguished by different stabilities and different solubilities.

The polymorphic forms of the invention are preferably substantially pure, meaning each polymorph form of Compound 1 includes less than 10%, preferably less than 5%, and preferably no more than 1% by weight of any one apparent impurity, including other polymorphic forms of the compound.

The polymorphic forms of the present invention may also exist together in a mixture. Mixtures of polymorphic forms of the present invention will have X-ray diffraction peaks characteristic of each of the polymorphs forms present in the mixture. For example, a mixture of two polymorphs will have a powder X-ray diffraction pattern that is a convolution of the X-ray diffraction patterns corresponding to the substantially pure polymorphs.

A. Polymorph Form A

Polymorph Form A of Compound 1 can be prepared as described in the above U.S. patent application Ser. No. 10/866,059.

Form A is characterized by an X-ray powder diffraction pattern with peaks at the following approximate diffraction angles (2θ), as shown in Table 1A below. 2θ angles shown in Table 1A below may range by about ±0.1 2θ.

TABLE 1A

| | Relative Intensity |
|---|---|
| Angle - 2 theta | (>9%) |
| 5.97897 | 50.22 |
| 9.10191 | 28.88 |
| 10.1028 | 100.0 |
| 11.6353 | 28.74 |
| 16.1237 | 37.33 |
| 17.9313 | 17.97 |
| 18.8624 | 12.50 |
| 20.0446 | 13.49 |
| 21.3785 | 15.76 |
| 25.0638 | 9.43 |
| 25.5830 | 12.30 |
| 26.0776 | 10.38 |

FIG. 1A provides an X-ray powder diffraction pattern for Form A.

The Raman spectra for Form A, shown in FIG. 1B, includes Raman shifts (cm⁻¹) having the values as shown in Table 1B below. Raman shifts shown in Table 1 B may range by about ±1 cm⁻¹.

TABLE 1B

| Raman shift | Relative Intensity |
|---|---|
| (cm⁻¹) | (>5%) |
| 615 | 8.7 |
| 712 | 6.5 |
| 785 | 7.0 |
| 825 | 5.0 |
| 948 | 7.0 |
| 982 | 6.0 |
| 1041 | 5.6 |
| 1249 | 48.6 |
| 1304 | 11.1 |
| 1340 | 16.8 |
| 1377 | 9.4 |
| 1431 | 11.4 |
| 1445 | 13.0 |
| 1507 | 14.1 |
| 1579 | 100 |
| 1632 | 12.4 |

The ¹³C SSNMR for Form A, shown in FIG. 1C, includes NMR peaks (ppm) and intensities as shown in Table 1C below. NMR peaks shown in Table 1C below may range by about ±1 ppm.

TABLE 1C

| Peak Number | Shift [ppm] | Int [au] |
|---|---|---|
| 1 | 159.7 | 1.84 |
| 2 | 158.0 | 2.08 |
| 3 | 154.3 | 1.52 |
| 4 | 152.3 | 1.79 |
| 5 | 148.3 | 4.72 |
| 6 | 147.0 | 4.00 |
| 7 | 143.6 | 3.97 |
| 8 | 139.2 | 5.91 |
| 9 | 136.6 | 5.39 |
| 10 | 135.4 | 9.69 |
| 11 | 131.4 | 12.0 |
| 12 | 129.8 | 5.67 |
| 13 | 121.9 | 7.43 |
| 14 | 119.1 | 0.28 |
| 15 | 110.5 | 3.26 |
| 16 | 96.2 | 1.43 |
| 17 | 94.3 | 1.95 |
| 18 | 48.8 | 2.33 |
| 19 | 45.9 | 2.57 |
| 20 | 14.8 | 9.14 |

The ¹⁹F SSNMR for Form A, shown in FIG. 1D, includes NMR peaks (ppm) and intensities as shown in Table 1D below. NMR peaks shown in Table 1D below may range by about ±1 ppm.

TABLE 1D

| Peak Number | Shift [ppm] | Int [au] |
|---|---|---|
| 1 | −114.6 | 12.0 |
| 2 | −116.5^{c} | ˜3.1 |
| 3 | −125.8 | 1.94 |
| 4 | −128.6^{c} | ˜3.4 |
| 5 | −130.6 | 12.0 |

Polymorph Form A is a meta-stable solvate form of Compound 1. Form A is chemically stable at 40° C. at 75% relative humidty for at least 6 weeks. A comparison of the initial X-Ray powder diffraction data for Form A, as shown in FIG. 1A, with the 6 week stability X-ray data indicates that Form A appears to be physically stable. However, a comparison of the initial DSC thermogram for Form A, as shown in FIG. 1E, against the 6 week DSC thermogram, as shown in FIG. 2G, indicates a small conversion of Form A to Form B. The initial DSC thermogram, FIG. 1E, indicates an endotherm onset at 167-171° C. at a scan rate of 10° C./min. The 6 week DSC thermogram, FIG. 2G, indicates an endotherm onset at 257° C. at a scan rate of 10° C./min. Thermal gravimetric analysis (TGA), as shown in FIG. 1F, indicates that Form A is a hydrated form.

B. Polymorph Form B

Polymorph Form B of Compound 1 can be prepared by slurring Form A in a suitable inert solvent and collecting the crystals therefrom. Suitable inert solvents include ethanol, methanol, isopropanol, toluene, benzene, or xylene isomers or mixtures thereof, among others. A specific solvent used in preparing Form B is toluene.

The preparation can be conducted at a temperature of about 70° C. to about 85° C., preferably at about 80° C. The aforesaid reaction is refluxed for a time period of at least 3 hours, and preferably for at least 5 hours. Seed crystals of polymorph Form B can be added to facilitate conversion. After refluxing, the solvent may be evaporated or filtered out.

The above procedure was originally used to convert polymorph Form A to Form B, but can be applied to convert other polymorphs to polymorph Form B, including as yet unidentified polymorphic forms. The procedure yields a highly crystalline, non-solvated anhydrous drug substance.

Form B is characterized by an X-ray powder diffraction pattern with peaks at the following approximate diffraction angles (2θ) as shown in Table 2A below. 2θ angles shown in Table 2A below may range by about ±0.1 2θ.

TABLE 2A

| | Relative Intensity |
|---|---|
| Angle - 2 theta | (>25%) |
| 10.499 | 92.29 |
| 11.281 | 31.88 |
| 11.960 | 100.00 |
| 12.499 | 66.92 |
| 13.558 | 88.21 |
| 15.400 | 65.64 |
| 16.363 | 37.45 |
| 19.042 | 42.34 |
| 20.452 | 35.28 |
| 22.298 | 94.84 |
| 25.355 | 38.08 |
| 26.667 | 54.24 |
| 29.1194 | 25.50 |

FIG. 2A provides an X-ray powder diffraction for Form B.

The Raman spectra for polymorph Form B, shown in FIG. 2B, includes Raman shifts as shown in Table 2B below. Raman shifts shown in Table 2B below may range by about ±1 cm⁻¹.

TABLE 2B

| Raman shift | Relative Intensity |
|---|---|
| (cm⁻¹) | (>2%) |
| 530 | 3.3 |
| 553 | 4.6 |
| 717 | 15.1 |
| 786 | 7.0 |
| 822 | 4.6 |
| 940 | 6.4 |
| 983 | 4.0 |
| 1040 | 3.6 |
| 1073 | 2.8 |
| 1114 | 5.2 |
| 1142 | 11.8 |
| 1246 | 34.0 |
| 1311 | 12.6 |
| 1333 | 38.4 |
| 1379 | 6.3 |
| 1428 | 12.5 |
| 1444 | 16.2 |
| 1514 | 12.8 |
| 1581 | 100 |
| 1632 | 17.0 |

The ¹³C SSNMR for polymorph Form B, shown in FIG. 2C, includes NMR peaks (ppm) and intensities as shown in Table 2C below. NMR peaks shown in Table 2C below may range by about ±1 ppm.

TABLE 2C

| Peak Number | Shift [ppm] | Int [au] |
|---|---|---|
| 1 | 157.7 | 3.33 |
| 2 | 155.8 | 4.44 |
| 3 | 153.0 | 1.79 |
| 4 | 150.9 | 4.21 |
| 5 | 149.3 | 3.60 |
| 6 | 147.1 | 5.40 |
| 7 | 146.2 | 3.78 |
| 8 | 144.5 | 3.30 |
| 9 | 143.3 | 3.16 |
| 10 | 141.4 | 8.88 |
| 11 | 137.8 | 6.96 |
| 12 | 136.2 | 10.1 |
| 13 | 129.6 | 12.0 |
| 14 | 124.6 | 3.62 |
| 15 | 121.6 | 7.06 |
| 16 | 118.8 | 2.06 |
| 17 | 107.9 | 3.48 |
| 18 | 99.4 | 1.69 |
| 19 | 95.9 | 2.45 |
| 20 | 92.7 | 1.77 |
| 21 | 46.9 | 1.56 |
| 22 | 42.9 | 2.25 |
| 23 | 15.0 | 5.26 |
| 24 | 12.4 | 7.51 |

The ¹⁹F SSNMR for polymorph Form B, shown in FIG. 2D, includes NMR peaks (ppm) and intensities as shown in Table 2D below. NMR peaks shown in Table 2D below may range by about ±1 ppm.

TABLE 2D

| Peak Number | Shift [ppm] | Int [au] |
|---|---|---|
| 1 | −113.9 | 11.5 |
| 2 | −118.6 | 9.40 |
| 3 | −124.3 | 7.70 |
| 4 | −126.2 | 12.0 |

Polymorph Form B is physically and chemically stable at 40° C. at 75% relative humidity for at least six weeks. Form B is believed to be the thermodynamically most stable form of currently identified polymorphs of Compound 1. Form B has an initial aqueous solubility greater than 20 mg/mL at pH=4, but after time the solution has been seen to gel. The DSC thermogram for Form B, as shown in FIG. 2E, indicates an endotherm onset at 248-250° C. at a scan rate of 10° C./min. Thermal gravimetric analysis (TGA) performed on Form B indicated that Form B is anhydrous.

C. Polymorph Form C

Polymorph Form C can be prepared by refluxing a suspension of Form A in a suitable inert solvent. Suitable solvents include tetrahydrofuran (THF), acetone, butyl acetate, ethyl acetate, heptane. A specific solvent for preparing Form C is THF.

The reaction is conducted at about room temperature. The aforesaid reaction is slurred a time period of at least 8 hours, and preferably overnight. After slurring, the solvent may be evaporated or filtered out.

Form C is characterized by an X-ray powder diffraction pattern with peaks at the following approximate diffraction angles (2θ) as shown in Table 3A below. 2θ angles shown in Table 3A below may range by about ±0.1 2θ.

TABLE 3A

| | Relative Intensity |
|---|---|
| Angle - 2 theta | (>18%) |
| 6.701 | 80.86 |
| 8.080 | 100.00 |
| 13.485 | 27.73 |
| 14.749 | 41.02 |
| 15.370 | 33.14 |
| 16.083 | 18.64 |
| 17.460 | 25.67 |
| 18.663 | 28.67 |
| 19.975 | 19.31 |
| 20.518 | 19.31 |
| 23.396 | 60.35 |
| 24.865 | 34.36 |
| 26.143 | 26.65 |

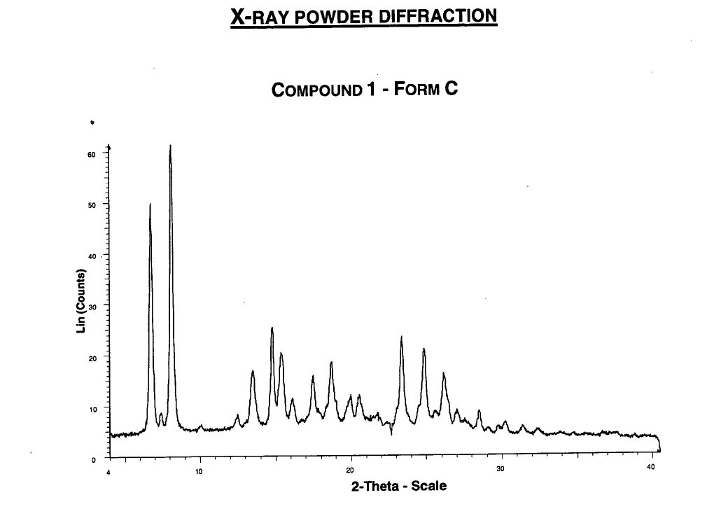

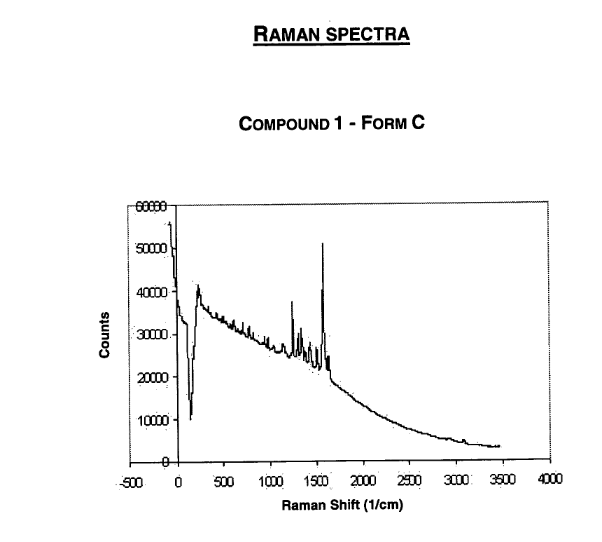

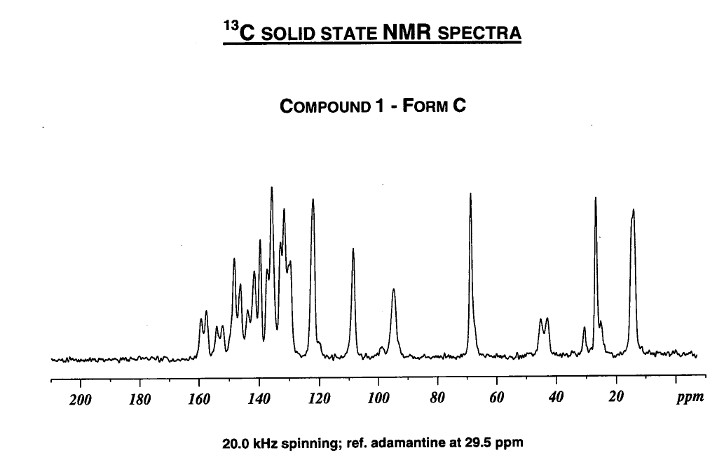

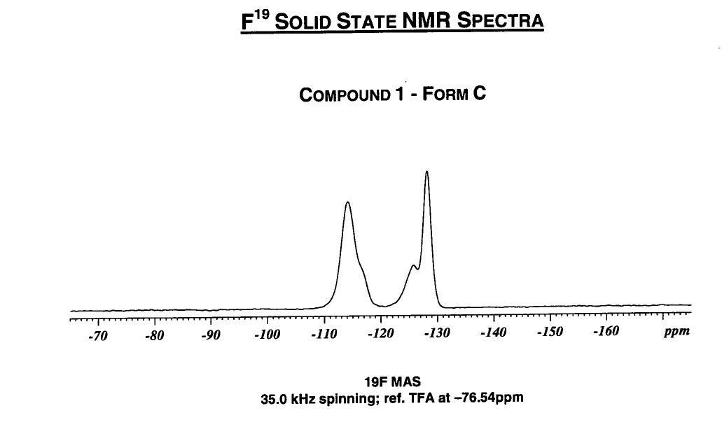

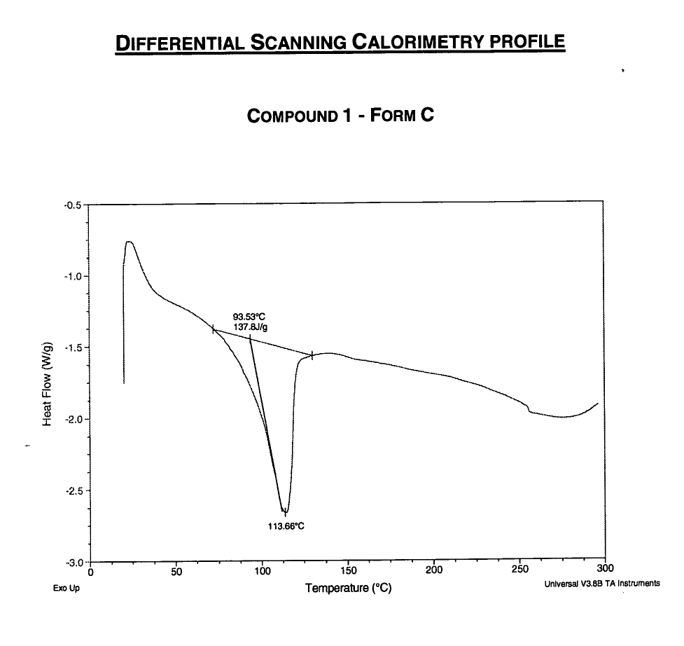

FIG. 3A provides an X-ray powder diffraction for Form C.

The Raman spectra for polymorph Form C, shown in FIG. 3B, includes Raman shifts as shown in Table 3B below. Raman shifts shown in Table 3B below may range by about +1 cm⁻¹.

TABLE 3B

| Raman shift | Relative Intensity |
|---|---|
| (cm⁻¹) | (>2%) |
| 617 | 8.0 |
| 713 | 9.2 |
| 781 | 9.4 |
| 826 | 5.5 |
| 945 | 7.5 |
| 982 | 8.3 |
| 1252 | 42.6 |
| 1304 | 15.3 |
| 1340 | 21.3 |
| 1381 | 7.1 |
| 1434 | 16.7 |
| 1446 | 11.7 |
| clai1510 | 15.9 |
| 1581 | 100 |
| 1630 | 12.1 |

The ¹³C SSNMR for polymorph Form C, shown in FIG. 3C, includes NMR peaks (ppm) as shown in Table 3C below. NMR peaks shown in Table 3C below may range by about ±1 ppm.

TABLE 3C

| Peak Number | Shift [ppm] | Int [au] |
|---|---|---|
| 1 | 159.3 | 2.45 |
| 2 | 157.5 | 3.04 |
| 3 | 154.1 | 1.89 |
| 4 | 152.1 | 1.95 |
| 5 | 148.2 | 6.86 |
| 6 | 146.1 | 4.95 |
| 7 | 143.8 | 3.07 |
| 8 | 141.5 | 5.88 |
| 9 | 139.6 | 8.17 |
| 10 | 137.2 | 6.05 |
| 11 | 135.7 | 12.0 |
| 12 | 132.9 | 7.98 |
| 13 | 131.6 | 10.4 |
| 14 | 129.6 | 6.64 |
| 15 | 122.0 | 11.1 |
| 16 | 108.5 | 7.52 |
| 17 | 98.8 | 0.33 |
| 18 | 94.8 | 4.53 |
| 19 | 68.7 | 11.46 |
| 20 | 45.1 | 2.26 |
| 21 | 43.0 | 2.32 |
| 22 | 30.7 | 1.66 |
| 23 | 26.8 | 11.1 |
| 24 | 25.3 | 2.08 |
| 25 | 14.0 | 10.2 |

The ¹⁹F SSNMR for polymorph Form C, shown in FIG. 3D, includes NMR peaks (ppm) as shown in Table 3D below. NMR peaks shown in Table 3D below may range by about ±1 ppm.

TABLE 3D

| Peak Number | Shift [ppm] | Int [au] |
|---|---|---|
| 1 | −114.1 | 9.35 |
| 2 | −116.4^{c} | ˜3.5 |
| 3 | −125.7 | 3.73 |
| 4 | −128.2 | 12.0 |

The DSC thermogram for Form C, as shown in FIG. 3E, indicates an endotherm onset at about 113° C., indicating that Form C is a meta-stable form.

D. Amorphous Form

The amorphous form of the invention can be prepared by dissolving any polymorph form in a suitable inert solvent, followed by removing the solvent by rotary evaporator under vacuum at a minimum temperature of about 35° C. within one half hour. Suitable solvents include tetrahydrofuran (THF), acetone, 2-butanone, butyl acetate, ethyl acetate. A specific solvent for preparing the amorphous form is THF. Specific conditions for making the amorphous form include rotary evaporation under vacuum at a temperature range of 40-50° C. within 5-10 minutes.

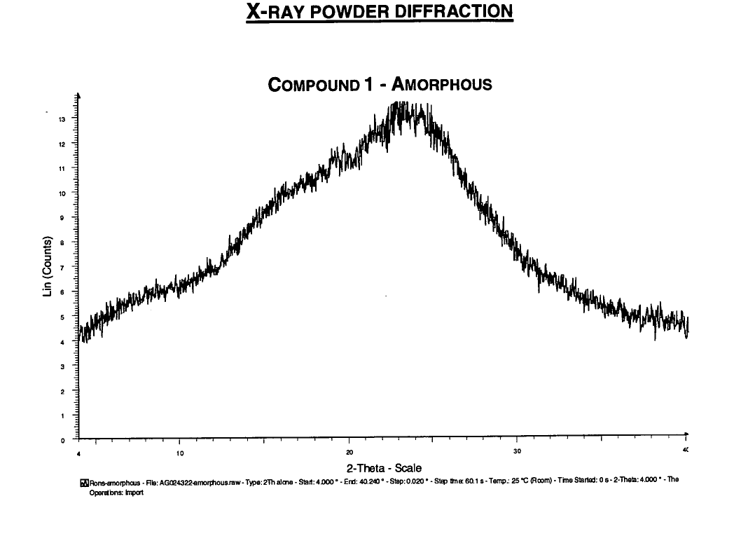

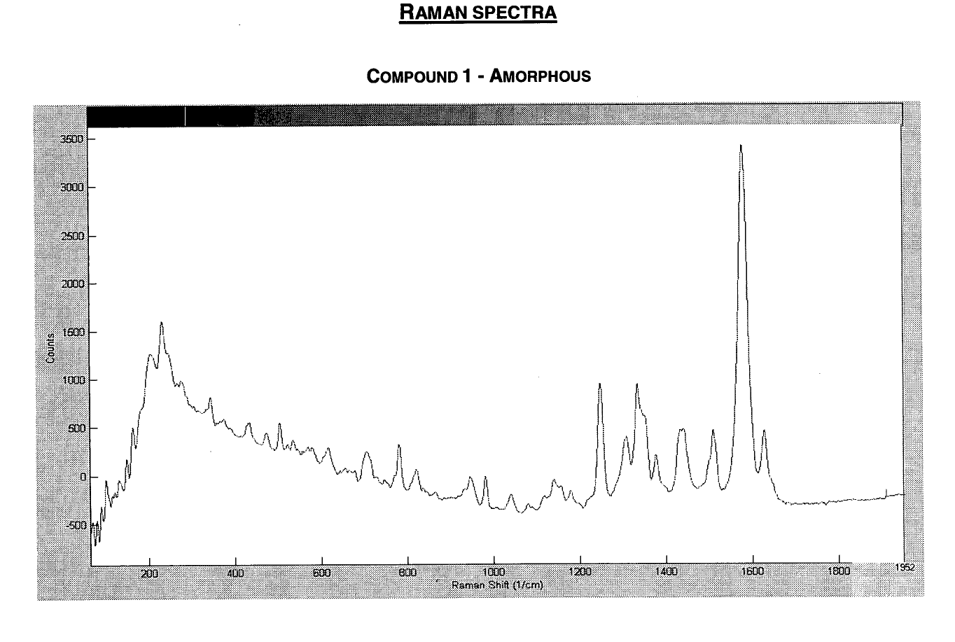

The amorphous form is characterized by a X-ray powder diffraction pattern as shown in FIG. 4A and by Raman spectra as shown in FIG. 4B. FIG. 4A is notable for a lack of isolated peaks. The Raman spectra for the amorphous form, shown in FIG. 4B, includes Raman shifts as shown in Table 4B below. Raman shifts shown in Table 4B below may range by about ±1 cm⁻¹.

TABLE 4B

| | Relative intensity |
|---|---|
| Raman shift (cm⁻¹) | >5% |
| 233 | 47.0 |
| 505 | 15.8 |
| 707 | 7.0 |
| 782 | 9.5 |
| 1249 | 27.7 |
| 1309 | 11.1 |
| 1335 | 27.3 |
| 1378 | 6.0 |
| 1511 | 13.3 |
| 1580 | 100.0 |
| 1629 | 13.2 |

III. Pharmaceutical Compositions of the Invention

The active agents (i.e., the polymorph or amorphous forms, or mixtures thereof, of Compound 1 described herein) of the invention may be formulated into pharmaceutical compositions suitable for both veterinary and human medical use. Pharmaceutical compositions of the invention comprise a therapeutically effective amount of the active agent and one or more inert, pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), and in “Handbook of Pharmaceutical Excipients”, Third Ed., Ed. A. H. Kibbe, Pharmaceutical Press, 2000. The active agents of the invention may be formulated in compositions including those suitable for oral, rectal, topical, nasal, ophthalmic, or parenteral (including intraperitoneal, intravenous, subcutaneous, or intramuscular injection) administration.

The amount of the active agent in the formulation will vary depending upon a variety of factors, including dosage form, the condition to be treated, target patient population, and other considerations, and will generally be readily determined by one skilled in the art. A therapeutically effective amount will be an amount necessary to modulate, regulate, or inhibit a protein kinase. In practice, this will vary widely depending upon the particular active agent, the severity of the condition to be treated, the patient population, the stability of the formulation, and the like. Compositions will generally contain anywhere from about 0.001% by weight to about 99% by weight active agent, preferably from about 0.01% to about 5% by weight active agent, and more preferably from about 0.01% to 2% by weight active agent, and will also depend upon the relative amounts of excipients/additives contained in the composition.

A pharmaceutical composition of the invention is administered in conventional dosage form prepared by combining a therapeutically effective amount of an active agent as an active ingredient with one or more appropriate pharmaceutical carriers according to conventional procedures. These procedures may involve mixing, granulating and compressing or dissolving the ingredients as appropriate to the desired preparation.

The pharmaceutical carrier employed may be either a solid or liquid. Exemplary solid carriers include lactose, sucrose, talc, gelatin, agar, pectin, acacia, magnesium stearate, stearic acid and the like. Exemplary liquid carriers include syrup, peanut oil, olive oil, water and the like. Similarly, the carrier may include time-delay or time-release materials known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an active agent is dissolved in an aqueous solution of an organic or inorganic acid, such as 0.3M solution of succinic acid or citric acid. If a soluble salt form is not available, the active agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. The composition may also be in the form of a solution of a salt form of the active agent in an appropriate aqueous vehicle such as water or isotonic saline or dextrose solution.

It will be appreciated that the actual dosages of the active agents used in the compositions of this invention will vary according to the particular complex being used, the particular composition formulated, the mode of administration and the particular site, host and disease being treated. Those skilled in the art using conventional dosage-determination tests in view of the experimental data for an agent can ascertain optimal dosages for a given set of conditions. For oral administration, an exemplary daily dose generally employed is from about 0.001 to about 1000 mg/kg of body weight, more preferably from about 0.001 to about 50 mg/kg body weight, with courses of treatment repeated at appropriate intervals. Administration of prodrugs is typically dosed at weight levels that are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

Proper formulation is dependent upon the route of administration chosen. For injection, the agents of the invention may be formulated into aqueous solutions, preferably in physiologically compatible buffers such as Hanks's solution, Ringers solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of Compound 1 and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be, for example, an ointment, vegetable oil, or an encapsulating material. A compound of the invention may also be injected directly into the vitreous and aqueous humor or subtenon.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

IV. Method of Using the Compounds of the Invention

The active polymorph agents of the invention may also be useful in the inhibition of the development of invasive cancer, tumor angiogenesis and metastasis.

Moreover, the active polymorph agents of the invention, for example, as inhibitors of the CDKs, can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like.

Compounds and compositions of the invention inhibit the kinase activity of, for example, CDK/cyclin complexes, such as those active in the G₀ or G₁ stage of the cell cycle, e.g., CDK2, CDK4, and/or CDK6 complexes.

A pharmaceutical composition according to the invention comprises a cell-cycle control agent and, optionally, one or more other active ingredients, such as a known antiproliferative agent that is compatible with the cell-cycle control agent and suitable for the indication being treated.

The compounds are useful as anti-angiogenesis agents and as agents for modulating and/or inhibiting the activity of protein kinases, thus providing treatments for cancer or other diseases associated with cellular proliferation mediated by protein kinases.

Therapeutically effective amounts of the agents of the invention may be used to treat diseases mediated by modulation or regulation of protein kinases. An “effective amount” is intended to mean that amount of an agent that, when administered to a mammal in need of such treatment, is sufficient to effect treatment for a disease mediated by the activity of one or more kinases. Thus, e.g., a therapeutically effective amount of a compound of the Formula I, salt, active metabolite or prodrug thereof is a quantity sufficient to modulate, regulate, or inhibit the activity of one or more kinases such that a disease condition which is mediated by that activity is reduced or alleviated.

“Treating” is intended to mean at least the mitigation of a disease condition in a mammal, such as a human, that is affected, at least in part, by the activity of one or more kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

In a specific embodiment of any of the inventive methods described herein, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In further specific embodiments of any of the inventive methods described herein, the method further comprises administering to the mammal an amount of one or more substances selected from anti-tumor agents, anti-angiogenesis agents, signal transduction inhibitors, and antiproliferative agents, which amounts are together effective in treating said abnormal cell growth. The compounds of the present invention may be combined with other anti-tumor agents, the methods of which are disclosed in WO038716, WO038717, WO038715, WO038730, WO038718, WO038665, WO037107, WO038786, WO038719, the contents of which are herein incorporated by reference in their entireties. Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as taxol (paclitaxel), docetaxel (Taxotere), 2′-N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethyinitrosourea, methylcyclohexyl-chloroethyinitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2,4-diamino-5-methyl-6-quinazolinyl)methyl]amino]benzoyl]-L-aspartic acid, N-[4-[[(2,4-diamino-5-ethyl-6-quinazolinyl)methyl]amino]benzoyl]-L-aspartic acid, N-[2-chloro-4-[[(2,4-diaminopteridinyl)methyl]amino]benzoyl]-L-aspartic acid, soluble Baker's antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadex™ (tamoxifen) or, for example anti-androgens such as Casodex™ (4′-cyano-3-(4-fluorophenylsulphony)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloprotienase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.). VEGF inhibitors, for example SU-5416 and SU-6668 (Sugen Inc. of South San Francisco, Calif., USA), can also be combined or co-administered with a compound of formula 1. VEGF inhibitors are described in, for example in WO 99/24440 (published May 20, 1999), PCT International Application PCT/IB99/00797 (filed May 3, 1999), in WO 95/21613 (published Aug. 17,1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following U.S. patent applications Ser. No. 09/221946 (filed Dec. 28, 1998); Ser. No. 09/454058 (filed Dec. 2, 1999); Ser. No. 09/501163 (filed Feb. 9, 2000); Ser. No. 09/539930 (filed Mar. 31, 2000); Ser. No. 09/202796 (filed May 22, 1997); Ser. No. 09/384339 (filed Aug. 26, 1999); and Ser. No. 09/383755 (filed Aug. 26, 1999); and the compounds disclosed and claimed in the following U.S. provisional patent applications 60/168207 (filed Nov. 30, 1999); 60/170119 (filed Dec. 10, 1999); 60/177718 (filed Jan. 21, 2000); 60/168217 (filed Nov. 30, 1999), and 60/200834 (filed May 1, 2000). Each of the foregoing patent applications and provisional patent applications is herein incorporated by reference in their entirety.

The compound of formula 1 may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

### V. EXAMPLES

The following examples are given to illustrate the invention, but should not be considered as limitations of the invention. Unless otherwise indicated, all temperatures are set forth in degrees Celsius and all parts and percentages are by weight.

### Example 1

### Preparation and Characterization of Polymorph Form A

The free base compound {5-[3-(4,6-Difluoro-1H-benzoimidazol-2-yl)-1H-indazol-5-yl]-4-methyl-pyridin-3-ylmethyl}-ethyl-amine (1.23 g), prepared as in Example 1 in U.S. patent application Ser. No. 10/866,069, was purified by trituration from cyclohexane to yield pure compound 1 (1.09 g, 74%) as an off-white solid.

FIG. 1A is an X-ray powder diffractogram of polymorph Form A of Compound 1. Form A was further characterized by Raman spectra, ¹³C solid state NMR, ¹⁹F solid state NMR, DSC profiling and TGA analysis. FIG. 1E is a DSC profile of a sample of Form A of Compound 1. Thermal gravimetric analysis (TGA) of Form A, as shown in FIG. 1F, indicates that Form A is a monohydrate, as determined by the 4.9% total weight loss upon an increase of temperature to 100° C.

### Example 2

### Preparation and Characterization Polymorph Form B

Polymorph Form B was prepared from Form A of Compound 1. A sample of Form A (20 mg) was slurried with 2 mL toluene in a round bottom flask. The flask was equipped with a mechanical stirrer, a reflux condenser and the mixture was heated at 80° C. for 5 hours. After cooling, the toluene was removed by helium evaporation to yield about 17 mg of Form B. HPLC analysis showed greater than 99.5% purity.

FIG. 2A is an X-ray powder diffractogram of polymorph Form B of Compound 1. Form B was further characterized by Raman spectra, ¹³C solid state NMR, ¹⁹F solid state NMR, DSC profiling and TGA analysis. Samples of Form B displayed an endotherm with onset at 250° C. Moreover, the DSC thermogram of Form A samples taken at 6 weeks of stability study at 40° C. at 75% relative humidity, as shown in FIG. 2G, revealed a new on-set melting point occurring at 257° C. indicating that a small amount of Form A had been converted to Form B.

### Example 3

### Preparation and Characterization of Polymorph Form C

Polymorph Form C was prepared from Form A of Compound 1. A sample of Form A (20 mg) was slurried in 2 mL THF at room temperature overnight. The slurry was stirred thoroughly. The THF was then removed by nitrogen evaporation to yield 17 mg of Form C.

FIG. 3A is an X-ray powder diffractogram of polymorph Form C of Compound 1. Form C was further characterized by Raman spectra, ¹³C solid state NMR, ¹⁹F solid state NMR, and DSC profiling. Samples of Form C displayed an endotherm with onset at 113° C. in the DSC thermogram, as shown in FIG. 3E, indicating that Form C is meta-stable.

### Example 4

### Preparation and Characterization of the Amorphous Form

The amorphous form was prepared from Form B of Compound 1. About 230 mg of Compound 1 was dissolved in 40 ml THF in a 100 ml round bottom flask at 40-50° C. (hot water bath). The solution was then rotary evaporated at the same temperature under lab vacuum (˜25 mmHg) for 5-10 minutes to produce the amorphous form.

FIG. 4A is an X-ray powder diffractogram of the amorphous form of Compound 1. The amorphous form was further characterized by Raman spectra as shown in FIG. 4B.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A crystalline form of 3{5-[3-(4,6-Difluoro-1H-benzoimidazol-2-yl)-1H-indazol-5-yl]-4-methyl-pyridin-3-ylmethyl}-ethyl-amine, 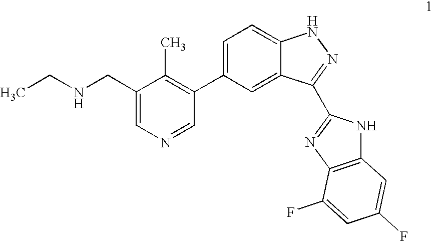
wherein the crystalline form is a substantially pure polymorph of Form B.

2. The crystalline form of claim 1, wherein the crystalline form has a powder X-ray diffraction pattern comprising peaks at diffraction angles (2θ) of 10.5±0.1, 12.0±0.1 and 22.3±0.1.

3. The crystalline form of claim 1, wherein the crystalline form has a powder X-ray diffraction pattern comprising peaks at diffraction angles (2θ) of 10.5±0.1, 12.0±0.1, 12.5±0.1, 13.6±0.1 and 22.3±0.1.

4. The crystalline form of claim 1, wherein the crystalline form has a powder X-ray diffraction pattern comprising peaks at diffraction angles (2θ) of 10.5±0.1, 12.0±0.1, 12.5±0.1, 13.6±0.1, 15.4±0.1 and 22.3±0.1.

5. The crystalline form of claim 1, wherein the crystalline form is characterized by Raman shifts (cm⁻¹) at 717±1, 1142±1 and 1514±1.

6. The crystalline form of claim 1, wherein the crystalline form is further characterized by Raman shifts (cm⁻¹) at 717±1, 1142±1, 1311±1, 1333±1 and 1514±1.

7. The crystalline form of claim 1, wherein the crystalline form is characterized by ¹³C solid state NMR shifts (ppm) at 118.8±1, 124.6±1, and 129.6±1.

8. The crystalline form of claim 1, wherein the crystalline form is characterized by ¹³C solid state NMR shifts (ppm) at 118.8±1, 124.6±1, 129.6±1, 155.8±1 and 157.7±1.

9. The crystalline form of claim 1, wherein the crystalline form is characterized by ¹⁹F solid state NMR shifts (ppm) at −113.9±1, −118.6±1, −124.3±1 and −126.2±1.

10. A crystalline form of 3{5-[3-(4,6-Difluoro-1H-benzoimidazol-2-yl)-1H-indazol-5-yl]-4-methyl-pyridin-3-ylmethyl}-ethyl-amine, represented by Formula 1 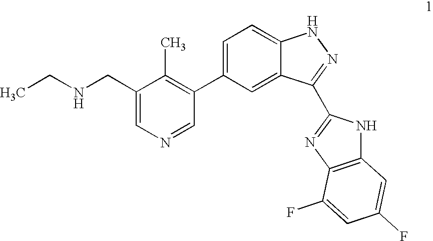
wherein the crystalline form is a substantially pure polymorph of Form C.

11. The crystalline form of claim 10, wherein the crystalline form has a powder X-ray diffraction pattern comprising peaks at diffraction angles (2θ) of 6.7±0.1 and 8.1±0.1.

12. The crystalline form of claim 10, wherein the crystalline form has a powder X-ray diffraction pattern comprising peaks at diffraction angles (29) of 6.7±0.1, 8.1±0.1 and 23.4±0.1.

13. The crystalline form of claim 10, wherein the crystalline form has a powder X-ray diffraction pattern comprising peaks at diffraction angles (2θ) of 6.7±0.1, 8.1±0.1, 14.7±0.1 and 23.4±0.1.

14. The crystalline form of claim 10, wherein the crystalline form is characterized by Raman shifts (cm⁻¹) of 1340±1, 1434±1 and 1510±1.

15. The crystalline form of claim 10, wherein the crystalline form is characterized by Raman shifts (cm⁻¹) of 1252±1, 1304±1, 1340±1, 1434±1 and 1510±1.

16. The crystalline form of claim 10, wherein the crystalline form is characterized by ¹³C solid state NMR shifts at 122.0±1, 135.7±1 and 139.6±1.

17. The crystalline form of claim 10, wherein the crystalline form is characterized by ¹³C solid state NMR shifts at 122.0±1, 131.6±1, 135.7±1, 139.6±1 and 148.2±1.

18. The crystalline form of claim 10, wherein the crystalline form is characterized by ¹⁹F solid state NMR shifts (ppm) at −114.1±1, −125.7≅1, and −128.2±1.

19. An amorphous form of 3{5-[3-(4,6-Difluoro-1H-benzoimidazol-2-yl)-1H-indazol-5-yl]-4-methyl-pyridin-3-ylmethyl}-ethyl-amine, characterized by having a powder X-ray diffraction pattern essentially the same as shown in FIG. 4A.

20. An amorphous form of 3{5-[3-(4,6-Difluoro-1H-benzoimidazol-2-yl)-1H-indazol-5-yl]-4-methyl-pyridin-3-ylmethyl}-ethyl-amine characterized by Raman shifts (cm⁻¹) of 233±1 and 1580±1.

21. An amorphous form of 3{5-[3-(4,6-Difluoro-1H-benzoimidazol-2-yl)-1H-indazol-5-yl]-4-methyl-pyridin-3-ylmethyl}-ethyl-amine characterized by Raman shifts (cm⁻¹) of 233±1, 1249±1, and 1580±1.

22. An amorphous form of 3{5-[3-(4,6-Difluoro-1H-benzoimidazol-2-yl)-1H-indazol-5-yl]-4-methyl-pyridin-3-ylmethyl}-ethyl-amine characterized by Raman shifts (cm⁻¹) of 233±1, 707±1, 1249±1, and 1580±1.

23. A mixture comprising at least one of the polymorphic forms B or C and an amorphous form of 3{5-[3-(4,6-Difluoro-1H-benzoimidazol-2-yl)-1H-indazol-5-yl]-4-methyl-pyridin-3-ylmethyl}-ethyl-amine.

24. A mixture of polymorphic forms of 3{5-[3-(4,6-Difluoro-1H-benzoimidazol-2-yl)-1H-indazol-5-yl]-4-methyl-pyridin-3-ylmethyl}-ethyl-amine, comprising at least two of the following polymorphic forms A, B, or C.

25. A pharmaceutical composition comprising the crystalline form of claim 1.

26. A pharmaceutical composition comprising the crystalline form of claim 10.

27. A pharmaceutical composition comprising the amorphous form of claim 19.

28. A pharmaceutical composition comprising the amorphous form of claim 20.

29. A pharmaceutical composition comprising the amorphous form of claim 21.

30. A pharmaceutical composition comprising the mixture of polymorphic forms of claim 22.

31. A method of treating a mammalian disease condition mediated by protein kinase activity, comprising administering to a mammal in need thereof a therapeutically effective amount of the pharmaceutical composition of any one of claims 25-30.

32. A method of treating a mammalian disease condition mediated by protein kinase activity, comprising administering to a mammal in need thereof a therapeutically effective amount of the crystalline form of claim 1.

33. The method of claim 31, wherein the mammalian disease condition is associated with unwanted angiogenesis or cellular proliferation.

34. A method of treating a hyperproliferative disorder mediated by CDK activity, comprising administering administering to a mammal in need thereof a therapeutically effective amount of a pharmaceutical composition which comprises any of the polymorphic forms of any of claims 1-18.

35. The method of claim 34, wherein the polymorphic form is administered in combination with a therapeutically effective amount of one or more substances selected from anti-tumor agents, anti-angiogenesis agents, signal transduction inhibitors, and antiproliferative agents.
